(19) United States
(12) Patent Application Publication
(10) Pub. No.: US 2023/0172442 A1
(43) Pub. Date: Jun. 8, 2023

(54) CLIP INDWELLING DEVICE AND CLIP

(71) Applicant: OLYMPUS MEDICAL SYSTEMS CORP., Tokyo (JP)

(72) Inventors: Tatsunori TSUNETO, Tokyo (JP); Shogo Shindo, Koganei-shi (JP)

(21) Appl. No.: 18/163,641

(22) Filed: Feb. 2, 2023

Related U.S. Application Data
(63) Continuation of application No. PCT/JP2020/030167, filed on Aug. 6, 2020.

(51) Int. Cl. A61B 1/018; A61B 1/005

(52) U.S. Cl. CPC A61B 1/018; A61B 1/0057

(57) ABSTRACT

A clip indwelling device includes an insertion portion; a power transmission member; a hollow member in which an insertion passage is formed; an arm member which includes a first and a second arms inserted into the insertion passage to protrude from a distal end of the hollow member, is connected to the power transmission member, and is constituted to switch between an open state and a closed state due to advance-retract movement of the power transmission member, and an adjustment mechanism which disposes the first and the second arms such that protrusion lengths of the first and the second arms in an axial direction with respect to the hollow member become equivalent to each other in the open state and disposes the first and the second arms such that the protrusion lengths thereof in the axial direction with respect to the hollow member differ from each other in the closed state.

The present invention relates to a clip indwelling device and a clip used for ligating tissue. This application is a continuation application based on International Patent Application No. PCT/JP2020/030167 filed on Aug. 6, 2020, and the content of the PCT international application is incorporated herein by reference.

### BACKGROUND

In the related art, a clip indwelling device is used for closing an opening generated in biological tissue or performing hemostatic treatment. A clip indwelling device is constituted to be inserted into the body using an endoscope device or a laparoscope and to be able to perform ligation by pinching a treatment target site with a clip. For example, Japanese Unexamined Patent Application, First Publication No. 2004-73646 discloses a ligation device that includes a pair of arms and a pipe member closing the pair of arms and performs ligation of biological tissue while retaining a state in which the biological tissue is pinched between distal end portions of the arms. In the ligation device of Japanese Unexamined Patent Application, First Publication No. 2004-73646, after tissue is ligated by closing distal ends of the pair of arms, coupling between an operation wire connected to the arms and the arms and connection between the pipe member and a coil sheath are canceled. As a result, a clip constituted of arms and a pipe member is separated from the ligation device and indwelled in tissue (mucous membrane).

### SUMMARY

For example, there are cases in which a plurality of clips are indwelled such that a treatment target site is surrounded. Since tissue to be indwelled a clip is soft, a pipe member does not stand upright by itself and falls on the tissue side after the clip is indwelled. When a plurality of clips are indwelled at positions closer to each other, if a pipe member of a clip which has been indwelled earlier falls, an operation of indwelling a next clip using a ligation device is hindered. For this reason, there is a need to perform treatment for changing a falling direction of the pipe member of a clip which has been indwelled earlier such that the pipe member vacates an indwelling position for a next clip, thereby a surgical operation time lengthens.

A clip indwelling device according to the present disclosure includes an insertion portion inserted into the body; a power transmission member provided so as to be advanced and retract inside the insertion portion; a hollow member attached to a distal end of the insertion portion and in which an insertion passage is formed in a central axis direction; an arm member including a first arm and a second arm inserted into the insertion passage so as to protrude from a distal end of the hollow member, is connected to the power transmission member, and is constituted to be able to switch between an open state in which the first arm and the second arm have been opened with respect to each other in response to advance-retract movement of the power transmission member and a closed state in which the first arm and the second arm have been brought closer to each other and closed with each other; and an adjustment mechanism configured to arrange the first arm and the second arm such that protrusion lengths of the first arm and the second arm with respect to the hollow member in an axial direction become equivalent to each other in the open state and disposes the first arm and the second arm such that the protrusion lengths thereof in the axial direction with respect to the hollow member differ from each other in the closed state.

A clip according to the present embodiment includes a hollow member in which an insertion passage is formed in a central axis direction; an arm member which includes a first arm and a second arm inserted into the insertion passage so as to protrude from a distal end of the hollow member, is connected to a power transmission member provided in a clip indwelling device, and is constituted to be able to switch between an open state in which the first arm and the second arm have been opened when relative positions of the first arm and the second arm in the central axis direction with respect to the hollow member are positions on a distal side of a first position and a closed state in which the first arm and the second arm have retracted inside the insertion passage from the first position and the first arm and the second arm are brought closer to each other and closed; and an adjustment mechanism which disposes the first arm and the second arm such that protrusion lengths of the first arm and the second arm in the central axis direction with respect to the hollow member become equivalent to each other in the open state and disposes the first arm and the second arm such that the protrusion lengths thereof in the central axis direction with respect to the hollow member differ from each other in the closed state.

A clip according to the present embodiment includes an arm member including a first arm elongated in a longitudinal direction and a second arm elongated in the longitudinal direction; and a hollow member including an insertion passage into which the arm member is inserted. The arm member is configured to switch between an open state in which the first arm and the second arm are opened with each other and a closed state in which the first arm and the second arm have been brought closer to each other and closed with each other. In the opened state, a protrusion length of the first arm with respect to the hollow member is same as a protrusion length of the second arm with respect to the hollow member in the longitudinal direction. In the close state, the protrusion length of the first arm with respect to the hollow member differs from the protrusion length of the second arm with respect to the hollow member in the longitudinal direction.

### BRIEF DESCRIPTION OF THE DRAWINGS

FIG. 1 is a general view of a clip indwelling device according to a first embodiment.

FIG. 2 is a partial cross-sectional view illustrating a distal end part of the clip indwelling device according to the first embodiment.

FIG. 3 is a partial cross-sectional view illustrating the distal end part of the clip indwelling device according to the first embodiment.

FIG. 4 is a cross-sectional view illustrating the distal end part of the clip indwelling device according to the first embodiment.

FIG. 5 is a cross-sectional view of a clip according to the first embodiment.

FIG. 6 is a cross-sectional view illustrating the distal end part of the clip indwelling device according to the first embodiment.

FIG. 7 is a side view of the clip according to the first embodiment.

FIG. 8 is a cross-sectional view illustrating a configuration of using the clip according to the first embodiment.

FIG. 9 is a schematic view illustrating a configuration of using the clip according to the first embodiment.

FIG. 10 is a cross-sectional view illustrating the distal end part of the clip indwelling device according to a second embodiment.

FIG. 11 is a cross-sectional view of a clip according to the second embodiment.

FIG. 12 is a cross-sectional view illustrating the distal end part of the clip indwelling device according to a third embodiment.

FIG. 13 is a front view illustrating an advance-retract movement guide member.

FIG. 14 is a cross-sectional view illustrating the distal end part of the clip indwelling device according to the third embodiment.

FIG. 15 is a cross-sectional view illustrating the distal end part of the clip indwelling device according to the third embodiment.

FIG. 16 is a cross-sectional view illustrating the distal end part of the clip indwelling device according to a fourth embodiment.

FIG. 17 is a cross-sectional view illustrating the distal end part of the clip indwelling device according to the fourth embodiment.

FIG. 18 is a cross-sectional view illustrating the distal end part of the clip indwelling device of a modification example of the fourth embodiment.

FIG. 19 is a cross-sectional view along line XIX-XIX in FIG. 18.

### DETAILED DESCRIPTION OF THE PREFERRED EMBODIMENTS

### First Embodiment

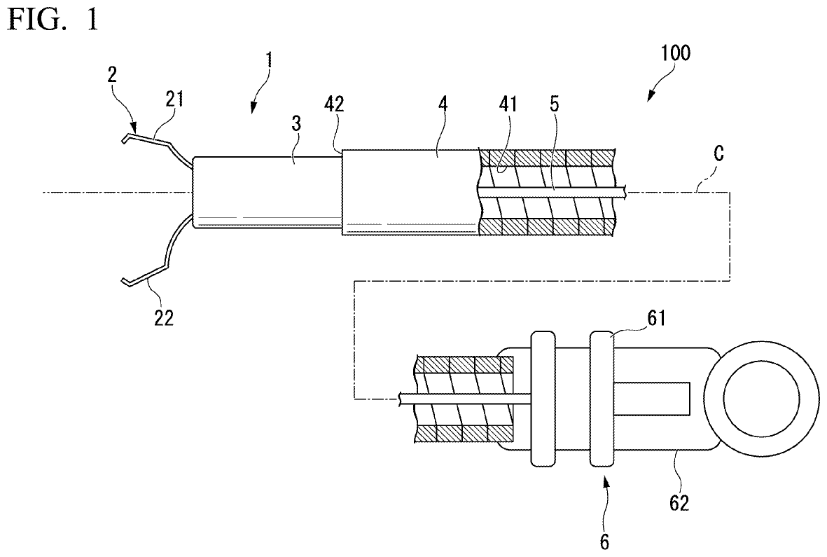

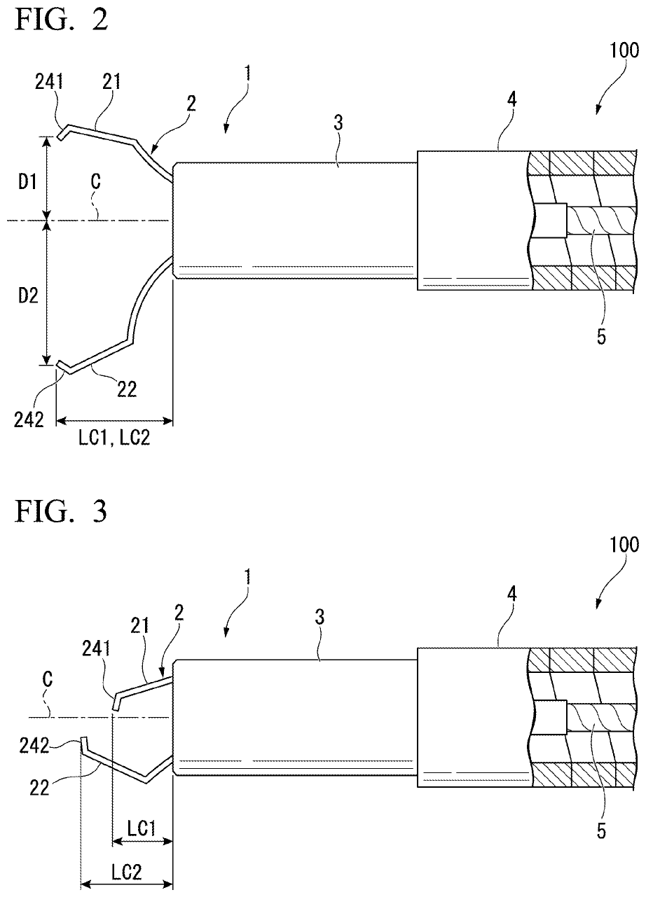

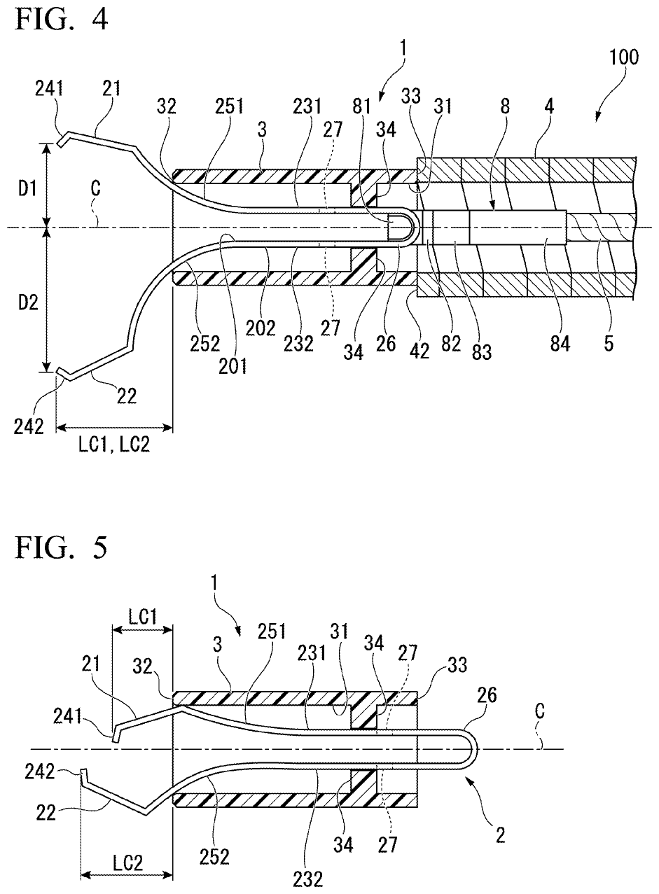

Hereinafter, a clip indwelling device 100 and a clip 1 according to a first embodiment will be described with reference to FIGS. 1 to 9. FIG. 1 is a general view illustrating the clip indwelling device 100 according to the present embodiment. FIGS. 2 and 3 are partial cross-sectional views illustrating a distal end part of the clip indwelling device 100. FIG. 4 is a cross-sectional view of the clip indwelling device 100 in a longitudinal direction. FIG. 5 is a cross-sectional view of the clip 1 in the longitudinal direction. FIG. 4 illustrates an open state of the clip 1, which will be described below. FIG. 5 illustrates a closed state of the clip 1, which will be described below.

As illustrated in FIG. 1, the clip indwelling device 100 includes a sheath 4, an operation wire 5, an operation portion 6, and the clip 1. The clip indwelling device 100 has a constitution in which the clip 1 is attached to a distal end of the sheath 4 by locking the clip 1 with a distal end of the operation wire 5 so that the clip 1 is capable of being indwelled in a treatment target site of a living body by an operation of the operation portion 6. The clip indwelling device 100 is a device in which the sheath 4 is inserted into the body and the clip 1 is indwelled so as to pinch tissue T of a treatment target site. For example, the clip indwelling device 100 according to the present embodiment is used by being inserted through a treatment tool channel of an endoscope and inserted into the body.

In the following description, in the longitudinal direction in a state in which the clip indwelling device 100 linearly extends, the operation portion 6 side will be referred to as a proximal side, and a side opposite to the proximal side and a side where the clip 1 is provided will be referred to as a distal side. A longitudinal axis of the sheath 4 is provided along a central axis C of a tubular holding tube 3 of the clip 1, which will be described below. In the following description, there are cases in which an axis of the clip indwelling device 100 extending in the longitudinal direction on the central axis C of the holding tube 3 is described as the central axis C.

The sheath 4 is an elongated tubular member having flexibility. In the sheath 4, a conduit 41 is formed throughout the overall length along the central axis C. For example, the sheath 4 is a coil sheath formed by tightly winding an element wire constituted of stainless steel such as SUS301 around the longitudinal axis. A proximal end of the sheath 4 is connected to an operation main body 62 of the operation portion 6. The conduit 41 opens at a distal end 42 of the sheath 4. The sheath 4 is an example of an insertion portion.

As illustrated in FIG. 1, the operation portion 6 includes the operation main body 62 and a slider 61. The slider 61 is provided such that it can slide in a direction of the central axis C with respect to the operation main body 62. The slider 61 is connected to the operation wire 5.

A proximal end of the operation wire 5 is fixed to the slider 61 of the operation portion 6, and a locking member 8 is fixed to the distal end of the operation wire 5. The operation wire 5 is a wire-like member formed using a metal solid wire or a stranded wire. The operation wire 5 is inserted through the inside of the sheath 4. The operation wire 5 is constituted to be capable of advancing and retracting inside the sheath 4 by advancing and retracting the slider 61 with respect to the operation main body 62. The operation wire 5 is an example of a power transmission member.

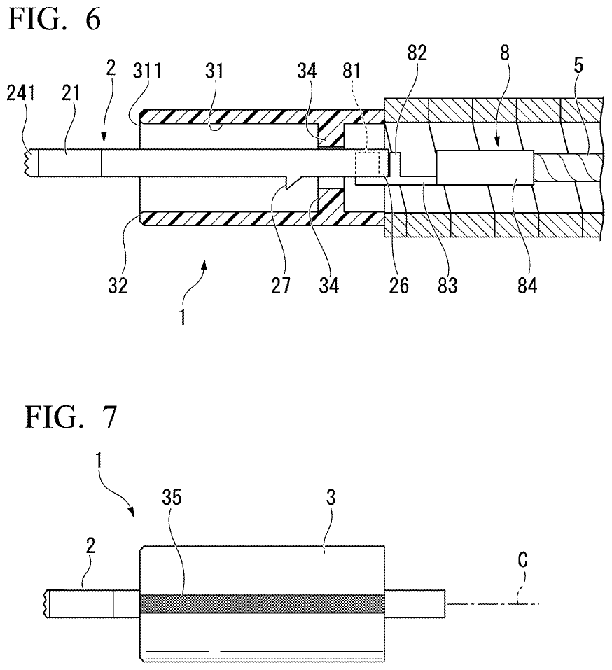

FIG. 6 is a cross-sectional view of the clip indwelling device 100 in the longitudinal direction and is a cross-sectional view in a direction orthogonal to FIG. 4. As illustrated in FIGS. 4 and 6, the locking member 8 has a fixed portion 84, a base portion 83, a proximal locking portion 82, and a distal locking portion 81. The fixed portion 84 is positioned in a proximal end portion of the locking member 8 and is fixed to the distal end of the operation wire 5. The base portion 83 is a flat plate-shaped part extending from a distal end of the fixed portion 84 to the distal side. The proximal locking portion 82 and the distal locking portion 81 protrude from the base portion 83 in a direction orthogonal to the central axis C. As illustrated in FIG. 4, a proximal portion of the distal locking portion 81 has a curved wall surface following the curved shape of a connection end portion 26 of an arm member 2. The proximal locking portion 82 is separated from the distal locking portion 81 to the proximal side and provided at a position facing the curved wall surface. The arm member 2 (which will be described below) is locked between the proximal locking portion 82 and the distal locking portion 81. The locking member 8 advances and retracts inside an insertion passage 31 in association with advance-retract movement of the operation wire 5.

The clip 1 is constituted to include the arm member 2 and the holding tube 3. The clip 1 is provided such that a proximal end of the holding tube 3 abuts the distal end of the sheath 4.

The arm member 2 has a first arm 21 and a second arm 22. For example, the arm member 2 is made using a thin and slender metal plate, such as stainless steel, a cobalt-chromium alloy, or a titanium. In the arm member 2, a U-shaped connection end portion 26 subjected to bending in a thickness direction is formed in an intermediate portion of the thin and slender plate, and both end portions of the plate constitute the first arm 21 and the second arm 22. In the following description, surfaces facing each other in the pair of arms 21 and 22 will be referred to as inner surfaces 201, and surfaces on sides opposite to the inner surfaces 201 and surfaces facing the insertion passage 31 will be referred to as outer surfaces 202.

The pair of arms 21 and 22 respectively include base portions 231 and 232, curved portions 251 and 252, and claw portions 241 and 242. The base portions 231 and 232 extend in a parallel manner on the distal side of the connection end portion 26. Stopper 27C is formed in each of the base portions 231 and 232. The stopper 27C is provided for fixing the arm member 2 to the holding tube 3 when the clip 1 is indwelled. As illustrated in FIG. 6, the stopper 27C is formed to protrude such that they extend in a direction intersecting the central axis C from both lateral sides of the base portions 231 and 232. The stopper 27C is formed to extend in a line-symmetrical manner with respect to the central axis C when viewed from the sides of the outer surfaces 202 of the arm member 2. Lengths of parts, in which the stopper 27C is provided, in a direction orthogonal to the central axis C are slightly greater than an opening diameter of a flange 34 of the holding tube 3.

In a natural state in which no external force is applied to the arm member 2, a curved shape is imparted to the arm member 2 such that a state in which the first arm 21 and the second arm 22 are opened is maintained. Parts to which a curved shape is imparted will be referred to as the curved portions 251 and 252.

The curved portions 251 and 252 are positioned on the distal sides of the base portions 231 and 232. The curved portions 251 and 252 are bendable such that they are separated from each other toward the distal sides in a natural state. In a natural state, the curved portions 251 and 252 have curvatures different from each other. The curvature of the curved portion 252 of the second arm 22 is greater than the curvature of the curved portion 251 of the first arm 21. The curved portions 251 and 252 having different curvatures are an example of an adjustment mechanism.

The claw portions 241 and 242 are provided on the distal sides of the curved portions 251 and 252. The claw portions 241 and 242 are formed in respective distal end portions of the arms 21 and 22 so as to be capable of being bent to the sides of the inner surfaces 201 in a direction in which they are brought closer to each other.

The holding tube 3 is a tubular hollow member in which the insertion passage 31 is formed in the direction of the central axis C. The insertion passage 31 communicates with the holding tube 3 from the distal end to the proximal end. The flange 34 is formed in a proximal end portion of the insertion passage 31. The flange 34 protrudes in an annular shape from an inner wall surface of the insertion passage 31 to an inward side in a radial direction. For example, the holding tube 3 is made using a metal material such as stainless steel, a titanium alloy (Ti-6AL-4V or the like), or a cobalt-chromium alloy; or a highly rigid resin material having moderate elasticity, such as polyphthalamide (PPA) or polyamide (PA). In a distal end portion of the insertion passage 31, a tapered surface 311 of which the opening diameter slightly expands toward a distal end surface of the holding tube 3 may be formed.

As illustrated in FIG. 7, a marker 35 is provided on an outer surface of the holding tube 3. The marker 35 is provided to indicate the position of the first arm 21 or the second arm 22. For example, the marker 35 is formed on the outer surface of the holding tube 3 by printing or the like. The marker 35 is a marker 35 having a slender shape extending along the central axis C of the holding tube 3. One marker 35 is formed in a part of the holding tube 3 in a circumferential direction. The position of the arm member 2 in the circumferential direction with respect to the holding tube 3 is subjected to positioning such that the first arm 21 or the second arm 22 is positioned on an extended line of the marker 35. Since the marker 35 is provided, when the clip is indwelled, a practitioner can recognize an image of an endoscope or the position of the first arm 21 or the second arm 22 around the central axis C.

The marker 35 may be formed at two places with an interval of 180 degrees in the circumferential direction of the holding tube 3. In this case, the arm member 2 is subjected to positioning with respect to the holding tube 3 such that the positions of the respective markers 35 and the positions of the first arm 21 and the second arm 22 coincide with each other in the circumferential direction.

As illustrated in FIG. 4, the connection end portion 26 is inserted into the insertion passage 31, and the connection end portion 26 is inserted into the insertion passage 31 between the proximal locking portion 82 and the distal locking portion 81, thereby being locked with the locking member 8. In a state in which the connection end portion 26 is locked with the locking member 8, the arm member 2 is inserted through the inside of the insertion passage 31 of the holding tube 3. As illustrated in FIG. 4, since the connection end portion 26 is disposed between the proximal locking portion 82 and the distal locking portion 81 and locked therewith, the arm member 2 is connected to the operation wire 5 via the locking member 8. As illustrated in FIGS. 4 and 6, the arm member 2 locked with the locking member 8 is retained such that the arm member 2 is capable of advancing and retracting along the central axis C inside the insertion passage 31.

The first arm 21 and the second arm 22 are inserted into the insertion passage 31 so as protrude from a distal end of the holding tube 3. That is, respective proximal portions of the base portions 231 and 232 of the first arm 21 and the second arm 22 are inserted into the insertion passage 31, and respective distal portions of the first arm 21 and the second arm 22 protrude from the distal end of the holding tube 3.

An opening of the flange 34 is slightly larger than external sizes of the locking member 8 and the connection end portion 26 in a locked state. As a result, the locking member 8 and the connection end portion 26 are locked with each other such that the flange 34 can pass therethrough while the locked state is maintained. The arm member 2 is provided such that it is capable of advancing and retracting from a position where the connection end portion 26 is disposed inside the insertion passage 31 to a position where the connection end portion 26 is exposed to the proximal end of the holding tube 3.

The arm member 2 is capable of advancing and retracting inside the insertion passage 31 of the holding tube 3 in the direction of the central axis C. The arm member 2 is constituted to be able to switch between the open state and the closed state in accordance with relative positions in the direction of the central axis C with respect to the holding tube 3. When the stoppers 27 are positioned on a distal side of the flange 34, the arm member 2 is capable of advancing and retracting with respect to the holding tube 3. Therefore, the clip 1 is constituted such that a so-called re-gripping operation can be performed. That is, if tissue is temporarily pinched by the pair of claw portions 241 and 242 of the arm member 2 and then the arm member 2 is relatively advanced with respect to the holding tube 3, the pair of arms 21 and 22 can be opened again, and a state in which the pair of claw portions 241 and 242 pinch tissue therebetween can be canceled. Moreover, if the arm member 2 is brought closer to tissue again and relatively retracted with respect to the holding tube 3 in a state in which the pair of arms 21 and 22 are opened, the tissue can be pinched again between the pair of claw portions 241 and 242.

When the stoppers 27 are positioned on the proximal side of the flange 34, the arm member 2 cannot advance and retract with respect to the holding tube 3. Specifically, if the stoppers 27 go over the flange 34 and move to the proximal side, the stoppers 27 are locked with a wall surface of the flange 34 on the proximal side so that advance movement of the stoppers 27 is restricted. In the closed state, since the arm member 2 is pinching the tissue T, the distal end portion of each of the arms 21 and 22 is retained in a state of protruding to a distal side of the holding tube 3. As a result, the arm member 2 is retained in the closed state.

As illustrated in FIG. 4, when the arm member 2 is disposed at an advance position on the distal side with respect to the holding tube 3 and at least parts of the curved portions 251 and 252 protrude to the distal side beyond the holding tube 3, the arm member 2 is in the open state. When the arm member 2 is in the open state, the connection end portion 26 is inserted into the insertion passage 31 and locked with the locking member 8 on the proximal side beyond the flange 34. During the open state, each of the curved portions 251 and 252 of the arm member 2 is positioned in the distal end portion of the insertion passage 31 of the holding tube 3. The outer surface of each of the curved portions 251 and 252 abuts the distal end portion of the insertion passage 31 of the holding tube 3. In the open state, the pair of arms 21 and 22 are separated from each other in a direction orthogonal to the central axis C.

As illustrated in FIG. 4, when the arm member 2 is in the open state, amounts of the first arm 21 and the second arm 22 protruding from the holding tube 3 in the direction of the central axis C are equivalent to each other. That is, when the arm member 2 is in the open state, the position of the distal end of the first arm 21 and the position of the distal end of the second arm 22 in the direction of the central axis C are substantially equivalent to each other.

The overall length of the second arm 22 is longer than the overall length of the first arm 21. Specifically, the length of the second arm 22 from the connection end portion 26 of the arm member 2 to the second claw portion 242 is longer than the length from the connection end portion 26 to the first claw portion 241. However, since the curvature of the curved portion 252 of the second arm 22 is greater than the curvature of the curved portion 251 of the first arm 21, a protrusion length LC1 of the first arm 21 from the holding tube 3 and a protrusion length LC2 of the second arm 22 from the holding tube 3 in the direction of the central axis C become equivalent to each other during the open state. That is, the curved portions 251 and 252 having different curvatures function as an adjustment mechanism.

When the arm member 2 is in the open state, a curved shape is imparted to the curved portion 252 such that the position of the distal end of the second arm 22 in the direction of the central axis C becomes a position substantially equivalent to the position of the distal end of the first arm 21. As a result of the protrusion lengths LC1 and LC2 of the first arm 21 and the second arm 22 from the holding tube 3 being equivalent to each other, when the clip 1 is indwelled, the arm member 2 in the open state is easily pressed against tissue in a treatment target site and tissue is easily pinched.

As illustrated in FIG. 2, when the arm member 2 is in the open state, a second distance D2 of the second arm 22 from the central axis C to the claw portion 242 in a direction orthogonal to the central axis C is longer than a first distance D1 of the first arm 21 from the central axis C to the first claw portion 241 in a direction orthogonal to the central axis C. As a result, while having a constitution in which the protruding amount of the second arm 22 is larger than the protruding amount of the first arm 21 in the closed state, they can be disposed such that the protrusion lengths LC1 and LC2 of the first arm 21 and the second arm 22 from the holding tube 3 become equivalent to each other in the open state. As a result, when the clip 1 is indwelled, the arm member 2 in the open state is easily pressed against tissue in a treatment target site and tissue is easily pinched.

As illustrated in FIG. 5, when the arm member 2 retracts from the open state with respect to the holding tube 3, the pair of arms 21 and 22 retract while being in contact with the distal end portion of the holding tube 3 and are brought closer to each other. If the arm member 2 retracts and the stoppers 27 go over the flange 34 and move to the proximal side beyond the flange 34, the arm member 2 is in the closed state. During the closed state, the connection end portion 26 of the arm member 2 protrudes to the proximal side beyond the proximal end of the holding tube 3. Further, each of the curved portions 251 and 252 of the first arm 21 and the second arm 22 retracts inside the insertion passage 31, and the claw portions 241 and 242 are brought closer to the central axis C. As described above, since the first arm 21 and the second arm 22 have different overall lengths, when the arm member 2 is in the closed state, the distal end of the first arm 21 and the distal end of the second arm 22 differ from each other in the protrusion lengths LC1 and LC2 in the direction of the central axis C with respect to the holding tube 3. Specifically, the protrusion length LC2 of the second arm 22 in the direction of the central axis C with respect to the holding tube 3 is longer than the protrusion length LC1 of the first arm 21 in the direction of the central axis C with respect to the holding tube 3. In the closed state, the first claw portion 241 is positioned on the proximal side of the second claw portion 242.

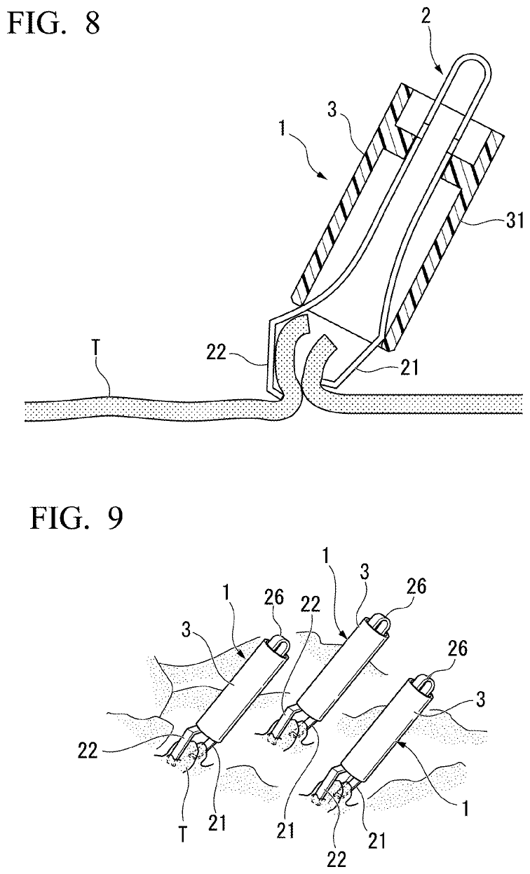

FIG. 8 is a schematic view illustrating a state in which the clip 1 is indwelled in a treatment target site. Generally, the tissue T having the clip 1 indwelled therein is soft. On the other hand, the clip 1 has a longer length in the direction of the central axis C than the holding tube 3 in the radial direction, and the center of gravity of the clip 1 at the time of indwelling is on the proximal side of the holding tube 3. For this reason, the clip 1 indwelled in the tissue T does not stand upright with respect to the tissue T, and it tilts. At this time, if a pair of arms are disposed and indwelled in a symmetrical manner as in a clip in the related art, a tilt direction of a holding tube is not settled. In contrast, in the clip 1 according to the present embodiment, as illustrated in FIG. 8, the second arm 22 protrudes longer than the first arm 21 and the tissue T is pinched therebetween during the closed state. As a result, the clip 1 tilts to the first arm 21 side where the protruding amount on the distal side is small. Namely, after indwelling, the tilt direction of the holding tube 3 of the clip 1 is settled to the first arm 21 side.

FIG. 9 is a schematic view illustrating a form of indwelling a plurality of clips 1 around a treatment target site. As illustrated in FIG. 9, at the time of ligation, the clips 1 are indwelled at positions closer to each other, and separation distances between the clips 1 may be shorter than lengths of the clips 1 in the direction of the central axis C. For this reason, when the clips are consecutively indwelled, if the holding tube of the clip which has been indwelled earlier falls at an indwelling position of a following clip, an indwelling operation will be hindered. However, in the clips 1 according to the present embodiment, in the closed state in which the tissue T is pinched, the tilt direction of the holding tube 3 is settled on the first arm 21 side. Therefore, a practitioner can easily perform adjustment such that the holding tube 3 of the clip 1 which has been indwelled earlier does not fall at the indwelling position of the clip 1 which will be indwelled next. Therefore, there is no need to perform an operation of changing the direction of a tilted holding tube as in clips in the related art, and thus a plurality of clips 1 can be smoothly indwelled. As a result, a surgical operation time can be shortened.

According to the clip indwelling device 100 and the clip 1 of the present embodiment, the direction in which the proximal end portion of the holding tube 3 of the clip 1 falls after indwelling can be controlled. Therefore, the proximal end portion of the clip 1 which has been indwelled earlier can be caused to tilt at a position where indwelling of a next clip is not hindered. As a result, when the plurality of clips 1 are consecutively indwelled at closer positions, the clip 1 which has been indwelled earlier does not hinder indwelling of the next clip 1 so that the plurality of clips can be smoothly indwelled.

According to the clip indwelling device 100 and the clip 1 of the present embodiment, in the open state, the curvature of the first curved portion 251 of the first arm 21 differs from the curvature of the second curved portion 252 of the second arm 22. As a result, the pair of arms 21 and 22 differ from each other in the amount of protrusion from the holding tube 3 in the closed state. However, the pair of arms 21 and 22 can be disposed such that the amounts of the pair of arms 21 and 22 protruding from the holding tube 3 become equivalent to each other in the open state. As a result, the pair of arms 21 and 22 are opened in the open state in a similar manner to those of clips in the related art so that it is easy to perform an operation of pressing and pinching the tissue T of a treatment target site.

According to the clip indwelling device 100 and the clip 1 of the present embodiment, the marker 35 is provided on the outer surface of the holding tube 3, and the position of the marker 35 is positionally aligned with the position of the arm member 2. As a result, the position around the central axis C of the arm member 2 and the direction of opening and closing the arm member 2 is capable of being recognized due to the marker 35.

When the marker 35 is provided in the holding tube 3, in a state in which the clip 1 is attached to the clip indwelling device 100, the positions of the pair of arms 21 and 22 around the central axis C are not particularly limited as long as they are positionally aligned with the position of the marker 35. For example, the clip 1 may be attached to the clip indwelling device 100 such that the second arm 22 is disposed at a part in which the distal end portion of the holding tube 3 is closest to the tissue T when the sheath 4 and the clip 1 are caused to advance with respect to the tissue T. That is, the clip 1 may be attached to the clip indwelling device 100 such that the second arm 22 is disposed on a lower portion side of the holding tube 3 when the sheath 4 is pushed to the tissue T. In this case, the clip 1 falls on the distal side of the sheath 4 in the advance-retract movement direction after the clip 1 is indwelled. As a result, if a plurality of clips 1 are indwelled in order from the distal side to the proximal side, a next clip 1 can be smoothly indwelled.

### Second Embodiment

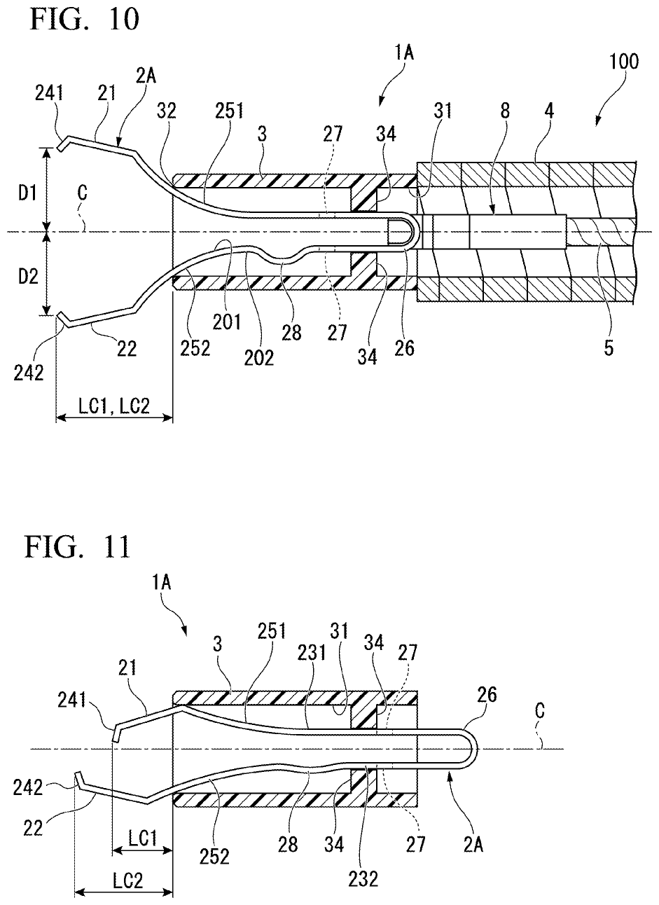

A clip 1A according to a second embodiment will be described with reference to FIGS. 10 and 11. In the following description, the same reference signs are applied to constituent elements common to those which have already been described, and duplicate description will be omitted. FIG. 10 illustrates the clip 1A in the open state, and FIG. 11 illustrates the clip 1A in the closed state.

The clip 1A according to the present embodiment is an example in which the constitution of the adjustment mechanism differs from that in the arm member 2 of the first embodiment. In the clip 1A, a telescopic portion 28 is provided in the base portion 232 of the second arm 22. As illustrated in FIG. 10, the telescopic portion 28 is curved in the thickness direction of the second arm 22. The curvatures of the curved portions 251 and 252 in the respective arms 21 and 22 of the clip 1A are substantially equivalent to each other.

As illustrated in FIG. 10, in the clip 1A in the open state, the protrusion length LC1 of the first arm 21 and the protrusion length LC2 of the second arm 22 from a distal end 32 of the holding tube 3 in the direction of the central axis C are equivalent to each other. In the open state, the first distance D1 of the first arm 21 from the central axis C to the first claw portion 241 in a direction orthogonal to the central axis C and the second distance D2 of the second arm 22 from the central axis C to the claw portion 242 in a direction orthogonal to the central axis C are equivalent to each other.

As illustrated in FIG. 11, if an arm member 2A retracts and the clip 1A is in the closed state, the curved state of the telescopic portion 28 is canceled due to elastic deformation. Specifically, if the arm member 2A retracts with respect to the holding tube 3 from a state in which the claw portions 241 and 242 of the pair of arms 21 and 22 press the tissue T in the open state, the pair of arms 21 and 22 are brought closer to each other. Since the tissue T is pinched between the claw portions 241 and 242, a state in which the distal end portions of the arms 21 and 22 are separated from each other wider than the opening of the insertion passage 31 is retained, and the distal end portions of the arms 21 and 22 do not enter the inside of the insertion passage 31 even if the arm member 2A retracts. In this state, if the arm member 2A continuously retracts, a pulling force is applied to both sides of the arm member 2A in the direction of the central axis C, the curved shape of the telescopic portion 28 is canceled and spread, and the length of the second arm 22 in the direction of the central axis C is increased. If the stoppers 27 go over the flange 34 to the proximal side, the stoppers 27 are locked with a proximal wall of the flange 34 so that advance movement of the arm member 2A is restricted. As a result of pinching the tissue T by the distal end portion of the arm member 2A, a state of being pinched by the tissue T and the distal end portion of the holding tube 3 is retained, thereby being in the closed state. As illustrated in FIG. 11, the protruding amount LC2 of the second arm 22 in the direction of the central axis C with respect to the holding tube 3 is greater than the protruding amount LC1 of the first arm 21 in the direction of the central axis C with respect to the holding tube 3 in the closed state. In the closed state, the first claw portion 241 is positioned on the proximal side of the second claw portion 242.

According to the clip 1A of the present embodiment, similar to the first embodiment, the direction in which the proximal end portion of the holding tube 3 of the clip 1 falls after indwelling can be controlled. Therefore, the proximal end portion of the clip 1 which has been indwelled earlier can be caused to tilt at a position where indwelling of a next clip is not hindered. As a result, when the plurality of clips 1 are consecutively indwelled at closer positions, the clip 1 which has been indwelled earlier does not hinder indwelling of the next clip 1 so that the plurality of clips can be smoothly indwelled.

### Third Embodiment

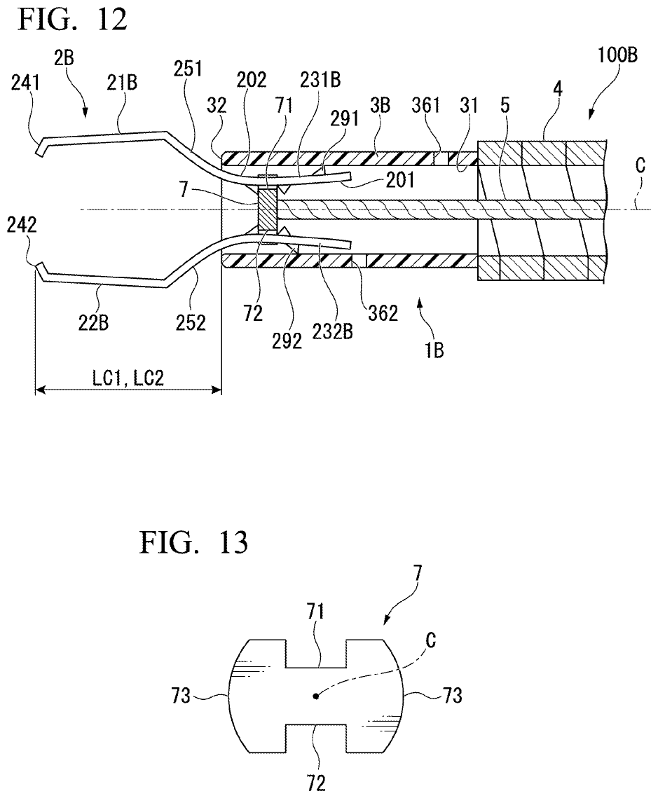

A clip indwelling device 100B and a clip 1B according to a third embodiment will be described with reference to FIGS. 12 to 15. The clip 1B according to the present embodiment differs from the first embodiment in constitutions of the arm member 2B and a holding tube 3B. The clip indwelling device 100B is provided with an advance-retract movement guide member 7, in place of the locking member 8 of the first embodiment.

FIG. 13 is a front view of the advance-retract movement guide member 7 viewed in the direction of the central axis C. The advance-retract movement guide member 7 has a flat plate shape, and a proximal surface is fixed to the operation wire 5. As illustrated in FIG. 13, the advance-retract movement guide member 7 has arc portions 73 and two recessed portions 71 and 72. The arc portions 73 have shapes imitating an inner circumferential surface of the insertion passage 31 and have dimensions allowing sliding contact with the inner circumferential surface of the insertion passage 31. Each of the two recessed portions 71 and 72 is a rectangular cutout portion. The advance-retract movement guide member 7 is provided so as to be capable of advancing and retracting inside the insertion passage 31 in association with advance-retract movement of the operation wire 5 with respect to the sheath 4. The advance-retract movement guide member 7 has dimensions allowing advance-retract movement such that the axis of the operation wire 5 becomes coaxial with the central axis C of the holding tube 3B at the time of advance-retract movement inside the insertion passage 31.

The holding tube 3B does not include the flange 34 of the holding tube 3 of the first embodiment and has a cylindrical shape. Two locking portions 361 and 362 are formed in the holding tube 3B. The locking portions 361 and 362 are holes penetrating the holding tube 3 toward the outer surface through the insertion passage 31 thereof The two locking portions 361 and 362 are opened 180 degrees apart in the circumferential direction of the holding tube 3. The positions of the two locking portions 361 and 362 differ from each other in the direction of the central axis C. The first locking portion 361 is formed on the proximal side of the second locking portion 362.

Configurations of the locking portions 361 and 362 are not limited to penetration holes. For example, the locking portions may be recessed portions allowing the stoppers 27 and 27 to enter the inside of the insertion passage 31 to be locked therewith.

A first arm 21B and a second arm 22B are independently constituted as separate bodies. The first arm 21B and the second arm 22B are separate members having substantially equivalent overall lengths. The first arm 21B and the second arm 22B have substantially equivalent shapes. The base portions 231B and 232B of the respective arms 21B and 22B have curved shapes.

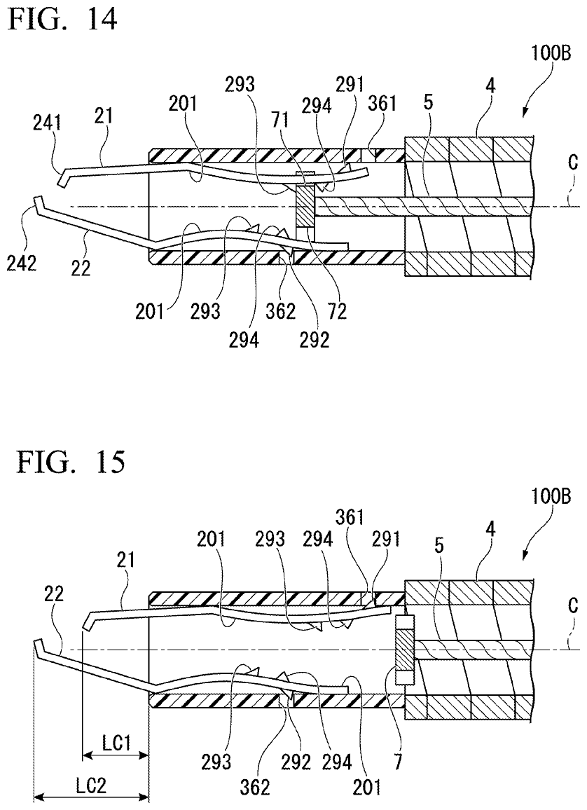

As illustrated in FIGS. 14 and 15, two locking pieces 293 and 294 are formed on the inner surfaces 201 of the base portions 231B and 232B of the respective arms 21B and 22B. The two locking pieces 293 and 294 are constituted to be capable of being locked with the advance-retract movement guide member 7. The two locking pieces 293 and 294 are separated from each other in the longitudinal direction and protrude in serrated shapes from the inner surfaces 201 of the base portions 231B and 232B in the thickness direction of the arms 21B and 22B. In the distal locking piece 293 on the distal side, a side on the proximal side is orthogonal to the inner surfaces 201 of the base portions 231B and 232B. The proximal locking piece 294 on the proximal side has a substantially triangular shape.

The separation distance between the distal locking piece 293 and the proximal locking piece 294 is slightly longer than the thickness of the advance-retract movement guide member 7. As illustrated in FIG. 12, the first arm 21B is positioned inside the first recessed portion 71, and the distal locking piece 293 and the proximal locking piece 294 are disposed on both sides of the first recessed portion 71 in the direction of the central axis C. The second arm 22B is positioned inside the second recessed portion 72, and the distal locking piece 293 and the proximal locking piece 294 are disposed on both sides of the recessed portions 71 and 72 of the advance-retract movement guide member 7 in the direction of the central axis C. In this state, since the pair of arms 21B and 22B are disposed inside the insertion passage 31, an arm member 2B is capable of advancing and retracting with respect to the holding tube 3B, and the arms 21B and 22B are attached to the operation wire 5 so as to be capable of being opened and closed.

Locking protrusions 291 and 292 are respectively provided in proximal portions of the first arm 21B and the second arm 22B. The locking protrusions 291 and 292 protrude in serrated shapes from the outer surfaces 202 of the arms 21B and 22B in the thickness direction of the arms 21B and 22B. The locking protrusions 291 and 292 are provided on the proximal side of the locking pieces 293 and 294. The first locking protrusion 291 of the first arm 21B is provided on the proximal side of the second locking protrusion 292 of the second arm 22B. The second locking protrusion 292 is provided in the vicinity of the proximal locking piece 294.

Since the locking pieces 293 and 294 are locked with the recessed portions 71 and 72 of the advance-retract movement guide member 7 on both sides of the direction of the central axis C, the arm member 2B is attached thereto so as to be capable of advancing and retracting in response to advance-retract movement of the operation wire 5. Although the details will be described below, the arm member 2B is capable of advancing and retracting with respect to the holding tube 3B until the locked state between the second arm 22B and the advance-retract movement guide member 7 is canceled. Therefore, the clip 1B is constituted to be able to perform so-called re-gripping.

The clip 1B in the open state will be described. During the open state, as illustrated in FIG. 12, in the direction of the central axis C, the protruding amount LC1 of the first arm 21B from the holding tube 3B and the protruding amount LC2 of the second arm 22B from the holding tube 3B become equivalent to each other. The locking protrusions 291 and 292 abut the inner circumferential surface of the insertion passage 31.

If the operation wire 5 retracts from the open state illustrated in FIG. 12, as illustrated in FIG. 14, the advance-retract movement guide member 7 retracts with respect to the holding tube 3B, and the locking protrusion 292 of the second arm 22B enters the inside of the second locking portion 362 first. As a result, the second arm 22B moves in a direction in which it is separated from the second recessed portion 72 of the advance-retract movement guide member 7 so that the locking state of the locking pieces 293 and 294 with respect to the second recessed portion 72 is canceled. Thereafter, even if the operation wire 5 retracts, retraction movement of the second arm 22B is restricted. When the operation wire 5 further retracts, the first locking protrusion 291 of the first arm 21B enters the first locking portion 361. Similar to the second arm 22B, the first arm 21B moves in a direction in which it is separated from the first recessed portion 71 of the advance-retract movement guide member 7 so that the locking state of the locking pieces 293 and 294 with respect to the first recessed portion 71 is canceled. As a result, as illustrated in FIG. 15, the arm member 2B is in the closed state.

A bent shape in the thickness direction is imparted to each of the first arm 21B and the second arm 22B. When the clip 1B is indwelled, the locking protrusions 291 and 292 are respectively locked with the locking portions 361 and 362 in a state in which the arms 21B and 22B pinch the tissue T therebetween. As a result, a force is applied to each of the arms 21B and 22B toward an outward side in the radial direction of the holding tube 3B, and the distal end portion of each of the arms 21B and 22B is retained in a state of protruding to the distal side of the holding tube 3. As a result, the arm member 2B is retained in the closed state.

When the arm member 2B is in the closed state, the distal end of the first arm 21B and the distal end of the second arm 22B differ from each other in the protruding amounts LC1 and LC2 in the direction of the central axis C with respect to the holding tube 3B. Specifically, the protruding amount LC2 of the second arm 22B in the direction of the central axis C with respect to the holding tube 3B is longer than the protruding amount LC1 of the first arm 21 in the direction of the central axis C with respect to the holding tube 3. In the closed state, the first claw portion 241 is positioned on the proximal side of the second claw portion 242. That is, the locking protrusions 291 and 292 and the locking portions 361 and 362 function as adjustment mechanisms.

As illustrated in FIG. 15, in the clip 1B after indwelling, similar to the foregoing embodiments, the distal end of the first arm 21B and the distal end of the second arm 22B differ from each other in the protruding amounts LC1 and LC2 in the direction of the central axis C with respect to the holding tube 3B. In the closed state, the protruding amount LC2 of the second arm 22 in the direction of the central axis C with respect to the holding tube 3 is longer than the protruding amount LC1 of the first arm 21 in the direction of the central axis C with respect to the holding tube 3. In the closed state, the first claw portion 241 is positioned on the proximal side of the second claw portion 242. As a result, similar to the foregoing embodiments, after indwelling, the tilt direction of the holding tube 3B is settled on the first arm 21B side.

According to the clip indwelling device 100B and the clip 1B of the present embodiment, similar to the foregoing embodiments, the direction in which the proximal end portion of the holding tube 3 of the clip 1 falls after indwelling can be controlled. Therefore, the proximal end portion of the clip 1 which has been indwelled earlier can be caused to tilt at a position where indwelling of a next clip is not hindered. As a result, when the plurality of clips 1 are consecutively indwelled at closer positions, the clip 1 which has been indwelled earlier does not hinder indwelling of the next clip 1 so that the plurality of clips can be smoothly indwelled.

### Fourth Embodiment

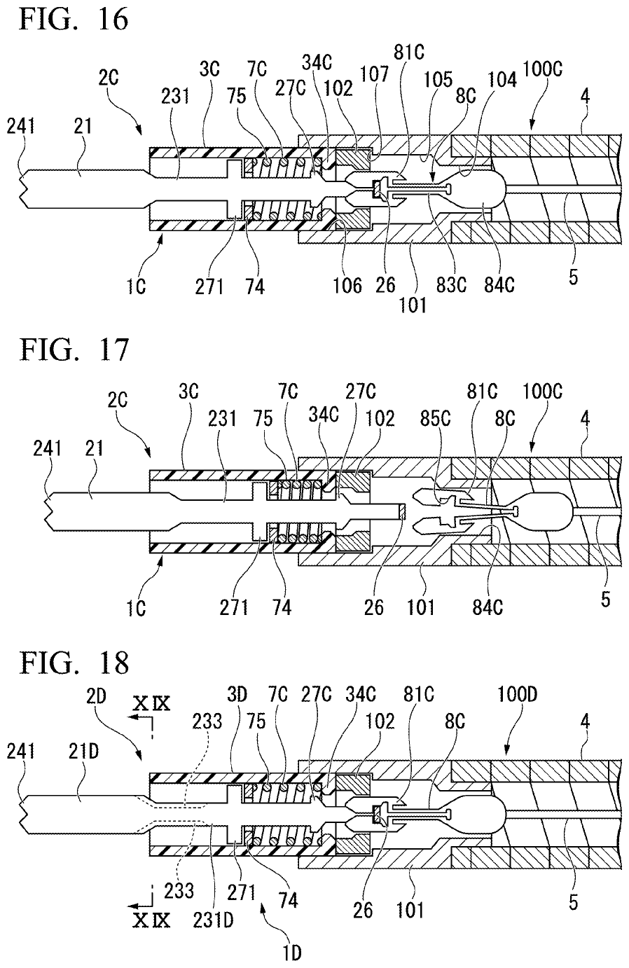

A clip indwelling device 100C and a clip 1C according to a fourth embodiment will be described with reference to FIGS. 16 to 19. Similar to each of the foregoing embodiments, the clip indwelling device 100C according to the present embodiment is a clip indwelling device 100C capable of performing a re-gripping operation using the clip 1C.

The clip indwelling device 100C is provided with a connection tube 101 at the distal end of the sheath 4. In the connection tube 101, a proximal tubular portion is inserted into the distal end of the sheath 4 and engaged therewith. A proximal opening of the connection tube 101 is provided so as to be in communication with the inside of the sheath 4, and the locking member and the operation wire 5 are inserted therethrough so as to be capable of advancing and retracting. In an internal conduit of the connection tube 101, step portions are provided such that opening dimension of the conduit sequentially increases, such as a first conduit 104, a second conduit 105, and a third conduit 106 in this order from the proximal side. The first conduit 104 has an opening dimension allowing the operation wire 5 and a fixed portion 84C of the locking member 8C to advance and retract. The third conduit 106 has an opening dimension allowing a holding tube 3C of the clip 1C (which will be described below) to be inserted thereinto. The opening dimension of the second conduit 105 is larger than that of the first conduit 104 and smaller than that of the third conduit 106. A step portion 107 is formed between the third conduit 106 and the second conduit 105. An opening of the second conduit 105 has a smaller diameter than the holding tube 3C. An auxiliary tube 102 is provided inside the third conduit 106. The auxiliary tube 102 is locked with the holding tube 3C so as to be rotatable around the central axis.

The locking member 8C has a fixed portion 84C, a base portion 83C, and distal locking portions 81C. The fixed portion 84C is fixed to the operation wire 5. The base portion 83C and the distal locking portions 81C have shapes of a pair of arms which can be opened and closed due to elastic deformation. A bent shape is imparted to the base portion 83C and the distal locking portions 81C in advance to be opened in a direction in which they are separated from each other in a natural state in which no external force is applied thereto. The base portion 83C and the distal locking portions 81C come closer to each other as they come into contact with the inner circumferential surface of the auxiliary tube 102 and are closed at a position along the central axis C of the holding tube 3.

Clip holding portions 85C are provided on the inward sides of each of the distal locking portions 81C. If the distal locking portions 81C and the base portion 83C are closed, a connection end portion 26 of an arm member 2C is pinched therein and the clip holding portions 85C are retained.

The clip 1C is provided with an advance-retract movement guide member 7C inside the holding tube 3C. The advance-retract movement guide member 7C is constituted to include a coil spring 75 and a washer 74.

The pair of arms 21 and 22 have a constitution similar to that of the first embodiment or the second embodiment. Moreover, spring stoppers 271 are formed in each of the pair of arms 21 and 22. Each of the spring stoppers 271 is formed on the distal side of the stopper 27C. The spring stoppers 271 protrude in a direction orthogonal to the central axis C from side surfaces of the arms 21 and 22.

The coil spring 75 is disposed inside the holding tube 3C such that a proximal end of the coil spring 75 abuts a distal surface of a flange 34C of the holding tube 3C. The washer 74 is disposed near a distal end of the coil spring 75. The base portions 231 and 232 of the pair of arms 21 and 22 are inserted into the openings of the coil spring 75 and the washer 74, and the spring stoppers 271 are disposed on the distal side of the advance-retract movement guide member 7C. The advance-retract movement guide member 7C is disposed between the spring stoppers 271 and the flange 34C of the holding tube 3C.

The proximal end of the holding tube 3C abuts the auxiliary tube 102 and is disposed inside the third conduit 106 of the connection tube 101. As illustrated in FIG. 16, in a state in which the coil spring 75 is not compressed, the arm member 2C is disposed at an advance position on the distal side with respect to the holding tube 3C. If the operation wire 5 is pulled, the spring stoppers 271 retract to the proximal side, the coil spring 75 is compressed, and the arms 21 and 22 move in a direction in which they are closed. Since the washer 74 is provided between the spring stoppers 271 and the coil spring 75, when the spring stoppers 271 retract and the coil spring 75 is compressed, the coil spring 75 can be prevented from being warped, and a winding diameter of the coil spring 75 can be maintained in a favorable state. As a result, operation of the coil spring 75 becomes stable, and the clip 1C can be stably retained on an extended line of the operation wire 5.

If the arms 21 and 22 retract and the stopper 27C comes into contact with a proximal end surface of the holding tube 3C and are locked therewith, the arms 21 and 22 are closed. When the arms 21 and 22 are closed and the stopper 27C is locked therewith, a locking member 8C retracts to the proximal side beyond the auxiliary tube 102, and the distal locking portions 81C are opened in a direction in which they are separated from each other. As illustrated in FIG. 17, if the distal locking portions 81C of the locking member 8C are opened, a state in which the connection end portion 26 is held by the distal locking portions 81C is canceled. As a result, the clip 1C deviates from the connection tube 101. If the distal locking portions 81C are opened in a natural state, the locking member 8C comes into contact with the first conduit 104 of the connection tube 101, and a resistance force is applied to a retraction movement operation of the operation wire 5. If the state in which the connection end portion 26 is held by the distal locking portions 81C is canceled and the distal locking portions 81C come into contact with the first conduit 104, a load during operation of the operation portion 6 changes so that an operator can recognize that the clip 1C is indwelled.

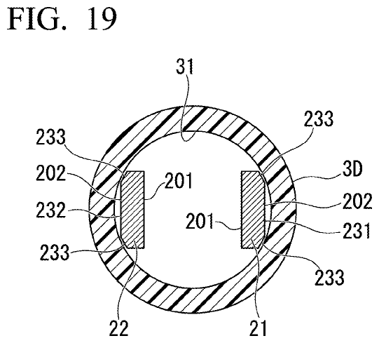

As in a clip 1D of a modification example illustrated in FIGS. 18 and 19, arms 21D and 22D may have chamfering surfaces 233 subjected to chamfering. FIG. 19 is a cross-sectional view along line XIX-XIX in FIG. 18. During opening/closing operation of the arms 21D and 22D, the arms 21D and 22D come into contact with an opening edge at a distal end of the insertion passage 31 of a holding tube 3D. The chamfering surfaces 233 are formed in regions where the arms 21D and 22D come into contact with the holding tube 3D. Specifically, as illustrated in FIG. 19, in each of the arms 21D and 22D, the chamfering surfaces 233 are formed in upper and lower end portions on the sides of the outer surfaces 202 of the base portions 231D and 232D. Due to the chamfering surfaces 233, sliding properties of the arms 21D and 22D with respect to the insertion passage 31 of the holding tube 3D are improved.

The chamfering surfaces 233 are not limited to the clip according to the fourth embodiment and may be provided in the clips of other embodiments.

The holding tube is not limited to the examples described in the foregoing respective embodiments. However, it need only be constituted such that an arm member is inserted therethrough and the arm member shifts from the open state to the closed state in accordance with the relative positions of the holding tube and the arm member.

In the clip indwelling devices according to the foregoing respective embodiments, an example in which the clip indwelling device is inserted into the body through a treatment tool channel of an endoscope and a clip is indwelled in tissue inside the body has been described, but the configuration of the clip indwelling device is not limited thereto. For example, it may be a device in which the insertion portion is constituted of a hard pipe and a clip is indwelled using a laparoscope.

Hereinabove, each of the embodiments of the present invention has been described, but the technical scope of the present invention is not limited to the foregoing embodiments. Within a range not departing from the gist of the present invention, combinations of the constituent elements in each of the embodiments can be changed, and each of the constituent elements can be subjected to addition of various changes or can be deleted. The present invention is not limited by the foregoing description and is limited only by the accompanying claims.

It is possible to provide a clip indwelling device and a clip in which a plurality of clips can be smoothly and consecutively indwelled and with which a surgical operation time can be shortened.

What is claimed is:

1. A clip indwelling device, comprising:
an insertion portion inserted into the body;
a power transmission member provided so as to be advanced and retract inside the insertion portion;
a hollow member attached to a distal end of the insertion portion and in which an insertion passage is formed in a central axis direction;
an arm member including a first arm and a second arm inserted into the insertion passage so as to protrude from a distal end of the hollow member, is connected to the power transmission member, and is constituted to be able to switch between an open state in which the first arm and the second arm have been opened with respect to each other in response to advance-retract movement of the power transmission member and a closed state in which the first arm and the second arm have been brought closer to each other and closed with each other; and
an adjustment mechanism configured to arrange the first arm and the second arm such that protrusion lengths of the first arm and the second arm with respect to the hollow member in an axial direction become equivalent to each other in the open state and disposes the first arm and the second arm such that the protrusion lengths thereof in the axial direction with respect to the hollow member differ from each other in the closed state.

2. The clip indwelling device according to claim 1,
wherein in the open state, a first distance between a distal end of the first arm and a central axis of the hollow member differs from a second distance between a distal end of the second arm and the central axis of the hollow member.

3. The clip indwelling device according to claim 1,
wherein the first arm has a first curved portion which is curved in the open state,
wherein the second arm has a second curved portion which is curved in the open state, and
wherein a curvature of the first curved portion and a curvature of the second curved portion differ from each other.

4. The clip indwelling device according to claim 1,
wherein the power transmission member is one wire-like member which is connected to the arm member, and
wherein the first arm and the second arm advance and retract inside the hollow member in association with advance-retract movement of the power transmission member inside the insertion portion.

5. The clip indwelling device according to claim 1,
wherein a telescopic portion is provided in the first arm or the second arm.

6. The clip indwelling device according to claim 1,
wherein the first arm and the second arm are independently provided in the arm member,
wherein an advance-retract movement guide member locked with a proximal end portion of the first arm and a proximal end portion of the second arm is provided at a distal end of the power transmission member,
wherein the hollow member includes a first locking portion for locking the proximal end portion of the first arm and a second locking portion for locking the proximal end portion of the second arm, and
wherein positions of the first locking portion and the second locking portion in the central axis direction differ from each other.

7. The clip indwelling device according to claim 1,
wherein the hollow member is provided with a positioning marker indicating a position of the arm member around the central axis.

8. A clip comprising:
a hollow member in which an insertion passage is formed in a central axis direction;
an arm member which includes a first arm and a second arm inserted into the insertion passage so as to protrude from a distal end of the hollow member, is connected to a power transmission member provided in a clip indwelling device, and is constituted to be able to switch between an open state in which the first arm and the second arm have been opened when relative positions of the first arm and the second arm in the central axis direction with respect to the hollow member are positions on a distal side of a first position and a closed state in which the first arm and the second arm have retracted inside the insertion passage from the first position and the first arm and the second arm are brought closer to each other and closed; and
an adjustment mechanism which disposes the first arm and the second arm such that protrusion lengths of the first arm and the second arm in the central axis direction with respect to the hollow member become equivalent to each other in the open state and disposes the first arm and the second arm such that the protrusion lengths thereof in the central axis direction with respect to the hollow member differ from each other in the closed state.

9. The clip according to claim 8,
wherein in the open state, a first distance between a distal end of the first arm and a central axis of the hollow member differs from a second distance between a distal end of the second arm and the central axis of the hollow member.

10. The clip according to claim 8,
wherein the first arm has a first curved portion which is curved in the open state,
wherein the second arm has a second curved portion which is curved in the open state, and
wherein a curvature of the first curved portion and a curvature of the second curved portion differ from each other.

11. The clip according to claim 8,
wherein a telescopic portion is provided in the first arm or the second arm.

12. The clip according to claim 8,
wherein the first arm and the second arm are independently provided in the arm member,
wherein the first arm includes a first locking portion locking a proximal end portion of the first arm with the hollow member,
wherein the second arm includes a second locking portion locking a proximal end portion of the second arm with the hollow member,
wherein positions of the first locking portion and the second locking portion differs from each other in the central axis direction.

13. The clip according to claim 8,
wherein the hollow member includes a marker showing a positon of the arm member around the central axis direction.

14. A clip comprising:
an arm member including a first arm elongated in a longitudinal direction and a second arm elongated in the longitudinal direction; and
a hollow member including an insertion passage into which the arm member is inserted,
wherein the arm member is configured to switch between an open state in which the first arm and the second arm are opened with each other and a closed state in which the first arm and the second arm have been brought closer to each other and closed with each other,
wherein in the opened state, a protrusion length of the first arm with respect to the hollow member is same as a protrusion length of the second arm with respect to the hollow member in the longitudinal direction, and
wherein in the close state, the protrusion length of the first arm with respect to the hollow member differs from the protrusion length of the second arm with respect to the hollow member in the longitudinal direction.

15. The clip according to claim 14,
wherein in the open state, a first distance between a distal end of the first arm and a central axis of the hollow member differs from a second distance between a distal end of the second arm and the central axis of the hollow member.

16. The clip according to claim 14 further comprising an adjustment mechanism configured to arrange the first arm and the second arm such that the protrusion lengths of the first arm and the second arm with respect to the hollow member in the longitudinal direction become equivalent to each other in a case where the arm member is in the open state; and the protrusion length of the first arm with respect to the hollow member differs from the protrusion length of the second arm with respect to the hollow member in the longitudinal direction in a case where the arm member is in the closed state.

17. The clip according to claim 16,
wherein the first arm has a first curved portion which is curved in the open state,
wherein the second arm has a second curved portion which is curved in the open state,
wherein a curvature of the first curved portion and a curvature of the second curved portion differ from each other, and
wherein the adjustment mechanism is the first curved portion and the second curved portion.

18. The clip according to claim 16,
wherein a telescopic portion curved in a thickness direction of the second arm is provided in the second arm,
wherein a curvature of the telescopic portion is released in the closed state of the arm portion, and
wherein the adjustment mechanism is the telescopic portion.

19. The clip according to claim 16,
wherein the first arm and the second arm are independently provided with the arm portion,
wherein the first arm includes a first locking portion locking a proximal end portion of the first arm with the hollow member,
wherein the second arm includes a second locking portion locking a proximal end portion of the second arm with the hollow member,
wherein positions of the first locking portion and the second locking portion differs from each other in the longitudinal direction, and
wherein the adjustment mechanism is the first locking portion and the second locking portion.

20. The clip according to claim 16,
wherein the hollow member includes a marker showing a positon of the arm member around the longitudinal direction.
